(19) United States
(12) Patent Application Publication
(10) Pub. No.: US 2026/0124801 A1
(43) Pub. Date: May 7, 2026

(54) METHOD OF PRECISION 3D PRINTED GLASSY CARBON

(71) Applicant: NANYANG TECHNOLOGICAL UNIVERSITY, Singapore (SG)

(72) Inventors: Xiao HU, Singapore (SG); Yong LU, Singapore (SG); Jingdan HU, Singapore (SG)

(21) Appl. No.: 19/117,567

(22) PCT Filed: Oct. 9, 2023
(86) PCT No.: PCT/SG2023/050680

§ 371 Date: Apr. 1, 2025

(30) Foreign Application Priority Data
Oct. 10, 2022 (SG) 10202251319A

(51) Int. Cl. B29C 64/124; B29C 35/02; B33Y 10/00; B33Y 70/00; C01B 32/05; C07C 255/54; C08F 2/00; C08F 120/30

(52) U.S. Cl. CPC B29C 64/124; B29C 35/02; C01B 32/05; C07C 255/54; C08F 2/00; C08F 120/30; B33Y 10/00; B33Y 70/00

(57) ABSTRACT

Disclosed herein are the synthesis and characterisations of the monoacrylate-functionalised phthalonitriles (PNs). Their chemical structures are verified with ¹H nuclear magnetic resonance (NMR) and Fourier transform infrared (FT-IR) spectra to confirm the successful synthesis. Subsequently, photopolymerisable resins, based on the prepared PN monomer, a commercial diacrylate and other additives can be formulated. Being post thermally treated, the PN based resins result in polymeric products with excellent thermal and mechanical performances, suggesting the high-performance nature of the PN based resins. Interestingly, the resins exhibit significant high carbon yield, indicating that they are greatly promising candidates as carbon precursors. As it is found that the resins have good three-dimensional (3D) printing capability and can be fabricated using high-resolution projection micro-stereolithography (PμSL) additive manufacturing technology, complex 3D structured objects are herein printed and performed follow-up post treatment. After being progressively pyrolysed up to 1000° C., the printed objects can be readily converted to glassy carbon (GC) structures with structural integrity and fidelity. Only ˜25% of shrinkage was found for the converted GC structure due to the significantly high char yield of the PN resins. This work not only expands the library of new class of high-performance PN resins for popular additive manufacturing but also features the precision 3D printing of GC with low shrinkage and high carbon yield for the first time.

### FIELD OF INVENTION

The current invention relates to novel compounds, resin precursor formulations for additive manufacturing which comprise the novel compounds, polymeric resin compositions obtained from the polymerisation of the novel compounds, methods of additive manufacturing using the resin precursor formulations, methods of providing a three-dimensional (3D) glassy carbon object, use of the novel compound in a method of additive manufacturing and in a method providing a glassy carbon object.

### BACKGROUND

The listing or discussion of a prior-published document in this specification should not necessarily be taken as an acknowledgement that the document is part of the state of the art or is common general knowledge.

As one of nature's most plentiful elements, carbon is the foundation of all organic and biological chemistry. Due to their unique characteristics, such as large surface area, low cost, consistent physicochemical properties, and interesting bioactivity, carbon-based materials constitute interesting alternatives in many scientific and technological fields. Numerous research efforts have allowed for the preparation of carbon-based materials to achieve products with particular electrochemical, thermal, mechanical and electrical properties.

Amongst various carbon materials, low-ordered and glass-like amorphous carbons that are obtained by the carbonization of some thermosetting resins under inert atmosphere are designated as glassy carbon (GC) or vitreous carbon, which is a hard and isotropic carbon material with high stiffness (Young's modulus of 21 GPa). GC is stiff, fragile and presents a surface fracture similar to that presented by glass. It presents excellent thermal and chemical stability, good impermeability to gases and liquids, good thermal and electrical conductivities and excellent biocompatibility. As a consequence, GC has been explored extensively in versatile technical applications (McCreery, R. L., Chem. Rev., 2008, 108, 2646), such as electrochemical sensors (Fan, S. et al., Chem. Pap., 2020, 74, 4411), energy storage devices (Islam, M. T. et al., Electrochim. Acta, 2020, 360, 136966), electrochemical devices for wastewater decontamination (Hsia, B. et al., Carbon, 2013, 57, 395), tools for precision moulding (Haq, M. R. et al., J. Micromech. Microeng., 2019, 29, 075010), ablative shields (Wang, S. et al., Polym. Degrad. Stabil., 2017, 144, 378), and biomedical implants and tissue engineering. Due to its good biocompatibility, GC may also be applied in many medical applications, such as heart valves, neural implants, and scaffolds for tissue regeneration.

The production of GC can be made from precursors like well-defined synthetic polymeric resins (e.g., phenolic and poly(furfuryl alcohol) resins) and biomass of plants (e.g., sucrose (Kubota, K. et al., Chem. Mater., 2020, 32, 2961), cellulose (Kaburagi, Y. et al., Carbon, 2005, 43, 2817), and tannin (Tondi, G. et al., Carbon, 2009, 47, 1480)). Precursor parameters such as molecular structures, weights, and aromatic contents govern the quality of final GC products.

In addition, it is known that specific geometries of GC can provide substantial advantages and benefits for advanced applications. However, the processing of GC materials is still a great challenge. Unlike metals, ceramics or polymers, GC can neither be melted, sintered nor polymerized due to its hard and fragile nature, therefore conventional processing methods such as screw extrusion, blending, roll pressing, injection moulding, welding, sintering cannot be used for obtaining complex (e.g. 3D) structures.

In recent years, with the rapid development of additive manufacturing (AM) (or 3D printing) technologies, the fabrication of non-processable materials has undergone unprecedented changes because very complex systems that could never be achieved (or with great difficulty) by conventional methods can be realized. The most-employed AM technologies include material extrusion printing (e.g. direct ink writing, DIW; fused deposition modelling, FDM), photopolymerisation printing (e.g. digital light processing, DLP); stereolithography, SLA), and selective laser sintering (SLS) printing. For carbonaceous material printing, the former two methods are mainly utilised and in particular, a wide variety of carbon based materials such as carbon fibres (Sanei, S. H. R. & Popescu, D., J. Compos. Sci., 2020, 4, 98), carbon black, carbon nanotubes (Acquah, S. F. A. et al., Carbon nanotubes and graphene as additives in 3D printing, in Carbon Nanotubes—Current Progress of Their Polymer Composites, M. R. Berber, I. H. Hafez (Eds.), InTech, 2016, DOI: 10.5772/63419), graphene, and graphene oxide (Guo, H. et al., Nano Mater. Sci., 2019, 1, 101) can be easily printed using DIW or FDM via a binder matrix. In contrast, less reports have been made on GC printing from high-performance carbon precursors. Bauer et al. reported the printing of GC nanolattice metamaterials via pyrolysis of a photocured resin (Bauer J. et al., Nature Materials, 2016, 15, 438). After pyrolysis, a GC nanostructure with 80% shrinkage was achieved and exhibited material strengths of up to 3 GPa, corresponding approximately to the theoretical strength of GC. In another work, Jacobsen et al. presented fabrication of open-cellular GC materials in a micro-scale with an approach to using a printed polymer template, which was impregnated with acrylonitrile and followed by pyrolysis (Jacobsen, A. J. et al., Carbon, 2011, 49, 1025). The resultant GC lattice structure showed a compressive modulus of 1.1 GPa and a failure strength of 10.2 MPa for a structure with relative density of 12.8%.

Although progress has been made, 3D printing of GC is still in its early stages of development. Challenges, such as adjusting the properties of the printed materials, increasing the carbon content of printing or printed materials, achieving a high-carbon content structure and reducing structure shrinkage after pyrolysis, etc., are yet to be addressed. Moreover, in contrast to the above mentioned carbonaceous materials, less attention has been paid to the 3D printing of GC arising from the lack of suitable and proper high-carbon yield precursors. Generally, the carbon yield of a carbon precursor for printed carbonaceous materials is less than 50% and the shrinkage is more than 50% after carbonization. Therefore, GC precursors with high carbon yield, good printability and retention of shape with low shrinkage during carbon formation are highly interesting and desirable for both academic research and industrial production due to their wide applications such as in high temperature fields, energy related applications, catalysis, and separation.

### SUMMARY OF INVENTION

We have surprisingly found that certain phthalonitrile (PN) compounds have unique properties that make them particularly suited to additive manufacture and the production of GC with a complex (e.g. 3D) structure. PN resins formed through the use of a suitable PN monomer, possibly along with a small amount of crosslinker(s), can be formulated into a high-performance resin for high-resolution SLA type 3D printing. The formulated resins exhibit good printability and are applicable to the high resolution AM technology, projection micro-stereolithography (PμSL). The PN based resins exhibit excellent thermal and mechanical properties with an achievable glass transition temperature (T_{g}) of around 300° C. and a modulus of 3.35 GPa, indicating their high-performance characteristics. After being thermally treated, the 3D printed structures can be subjected to a carbonisation process. Eventually, GC products with complex 3D structures can be obtained. As such, the resins disclosed herein enable GC products with a complex structure to be formed, these GC structures may be formed with ultra-high carbon yield and ultra-low structure shrinkage following conversion from the resultant (e.g. PμSL) printed structures. These disclosures offer an avenue to achieve complex high-resolution GC structures with AM technology, enabling their practical use in advanced applications.

Aspects and embodiments of the invention are described in the following numbered clauses.
1. A compound of formula I:

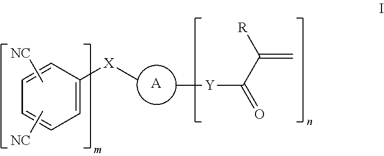

wherein:
m and n are independently from 1 to 3;
X is a bond, O or NH;
R is H or an aliphatic group; and
A is an aromatic, a heterocyclic or an aliphatic group and is unsubstituted
or is substituted by one of more of the group consisting of halo, OR^{1a}, and NR^{1b}R^{1c};
R^{1a} to R^{1c} are each independently selected from H and C₁ to C₁₀ alkyl; and
Y is a bond, a C₁ to C₁₀ alkylene group or a
—C₁ to C₁₀ alkylene-O— group, where in the latter group the alkylene portion is directly attached to A and the oxygen atom is directly attached to the carbon of the C═O double bond.
2. The compound according to Clause 1, wherein A is an aromatic or aliphatic group and is unsubstituted or is substituted by one of more of the group consisting of OR^{1a}, and NR^{1b}R^{1c}.
3. The compound according to Clause 2, wherein A is phenyl and is unsubstituted or is substituted by one of more of the group consisting of OR^{1a}.
4. The compound according to any one of the preceding clauses, wherein R^{1a} to R^{1c}, where present, are H or CH₃.
5. The compound according to any one of the preceding clauses, wherein X is NH or O, optionally wherein X is O.
6. The compound according to any one of the preceding clauses, wherein n and m are 1.
7. The compound according to any one of the preceding clauses, wherein Y is a
   —C₁ to C₃ alkylene-O— group, where the alkylene portion is directly attached to A and the oxygen atom is directly attached to the carbon of the C═O double bond.
8. The compound according to any one of the preceding clauses, wherein R is H or CH₃, optionally wherein R is H.
9. The compound according to any one of the preceding clauses, wherein the compound of formula I is selected from:

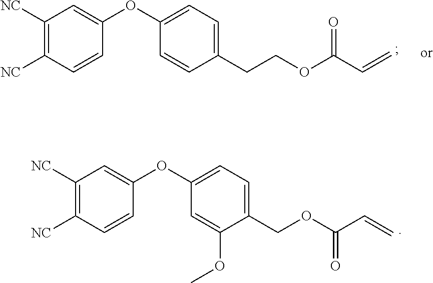 [SMILES: C=CC(=O)OCCc1ccc(Oc2ccc(C#N)c(C#N)c2)cc1 ; C=CC(=O)OCc1ccc(Oc2ccc(C#N)c(C#N)c2)cc1OC]

10. A resin precursor formulation for additive manufacturing, comprising:
   a compound of formula I as defined in any one of Clauses 1 to 9;
   a photoinitiator; and
   an organic solvent suitable for use in additive manufacturing.
11. The resin precursor formulation according to Clause 10, wherein the formulation further comprises one or more of a crosslinking agent (e.g. a diacrylate or a polyacrylate (e.g. from 2 to 5 acrylate groups), such as bisphenol A ethoxylate diacrylate), a photoabsorber (e.g. Sudan I), and a filler (e.g. an organic filler and/or an inorganic filler).
12. The resin precursor formulation according to Clause 10 or Clause 11, wherein the formulation is suitable for use in a stereolithography-type additive manufacturing process, such as stereolithography and/or digital light processing.
13. The resin precursor formulation according to any one of Clauses 10 to 12, wherein one or more of the following apply:
   (i) the photoinitiator is selected from one or more of the group consisting of 2,4,6-trimethyl benzoyldiphenyl phosphine oxide, 1-hydroxycyclohexyl phenyl ketone, 2-hydroxy-2-methylphenyl-propane-1-one, and, more particularly, phenylbis(2,4,6-trimethylbenzoyl)phosphine oxide); and
   (ii) the organic solvent is selected from one or more of the group consisting of toluene, chloroform, tetrahydrofuran, and more particularly, toluene, dimethylformamide, and N-methylpyrrolidine, optionally wherein the organic solvent is N-methylpyrrolidine.
14. The resin precursor formulation according to any one of Clauses 10 to 13, wherein the resin precursor formulation comprises:
   a compound of formula I as defined in any one of Clauses 1 to 9;
   a photoinitiator;
   an organic solvent suitable for use in additive manufacturing;
   a crosslinking agent; and
   a photoabsorber,
   optionally wherein: the resin precursor formulation comprises:
   a compound of formula I as defined in any one of Clauses 1 to 9;
   phenylbis(2,4,6-trimethylbenzoyl)phosphine oxide);
   N-methylpyrrolidine;
   bisphenol A ethoxylate diacrylate; and
   Sudan I.
15. A polymeric resin composition obtained from the polymerisation of a compound of formula I as described in any one of Clauses 1 to 9.
16. The polymeric resin composition according to Clause 15, wherein the polymeric resin composition has one or more of the following properties:
   (a) a T_{d} that is greater than or equal to 400° C.;
   (b) a T_{g} that is greater than or equal to 300° C.;
   (c) a bending strength that is greater than or equal to 100 MPa, such as greater than or equal to 150 MPa;
   (d) a modulus greater than or equal to 3.5 Gpa;
   (e) a carbon yield, following a sintering process, of greater than or equal to 60%; and
   (f) a shrinkage value of from 20 to 40%, such as from 25 to 30%, for a glassy carbon three-dimensional object relative to a green polymeric resin three-dimensional product following a sintering process to provide the glassy carbon three-dimensional product.
17. The polymeric resin composition according to Clause 15 or Clause 16, wherein the polymeric resin has been formed into a three-dimensional object.
18. A process of additive manufacturing, the process comprising:
   (ai) providing a resin precursor formulation for additive manufacturing as described in any one of Clauses 10 to 12; and
   (aii) controlling a stereolithography-type apparatus to form a three-dimensional object by using the resin precursor formulation, wherein the resin precursor formulation is deposited by the apparatus on a surface in a layer by layer operation and each layer is subjected to a polymerisation reaction before each subsequent layer is laid down.
19. A process of providing a three-dimensional glassy carbon object, the process comprising the steps of:
   (bi) providing a three-dimensional object comprising a polymeric resin material obtained from the polymerisation of a compound of formula I as described in any one of Clauses 1 to 9; and
   (bii) subjecting the three-dimensional object to carbonisation through the application of heat to provide a glassy carbon object.
20. Use of a compound of formula I as described in any one of Clauses 1 to 9 in a method of additive manufacture.
21. Use of a compound of formula I as described in any one of Clauses 1 to 9 in a method providing a glassy carbon object.

### DRAWINGS

FIG. 1 depicts the generic structures of phthalonitrile (PN) precursors for precision carbon 3D printing.

FIG. 2 depicts the preparation approach to the photopolymerisable PN monomers, PN-T and PN-V.

FIG. 3 depicts nuclear magnetic resonance (NMR) spectrum of acrylated PN monomers (a) PN-T and (b) PN-V.

FIG. 4 depicts Fourier transform Infrared (FT-IR) spectra of PN-T-1 and (b) PN-V-1.

FIG. 5 depicts (a) FT-IR spectra of monomer PN-T and resin PN-T-1 before and after thermal treatment; (b) thermogravimetric analysis (TGA) thermogram; (c) flexure stress-strain curves; and (d) storage modulus and tan δ plotted as a function of temperature for resin PN-T-1 and PN-V-1.

FIG. 6 depicts (a) from left to right, structures and shrinkage (%) of as-printed, thermally treated and carbonized honeycomb structures using PN-V-1 resin; and (b) scanning electron microscopy (SEM) images of top surface of printed GC honeycomb structures.

FIG. 7 depicts (a) SEM images and elemental mapping of cross-section of printed GC honeycomb structures using PN-V-1 resin; and (b) Raman spectrum of resulted GC product.

FIG. 8 depicts examples of as-printed and thermally treated PN structures as well as converted GC products.

FIG. 9 depicts a schematic process of PμSL printing, post-treatment and GC conversion.

FIG. 10 depicts (a) PμSL assisted 3D printed, thermally treated and pyrolyzed honeycomb structures using PN-V-1 resins; (b) (c) (d) SEM of top surface and internal surface; (e) Raman spectra; (f) X-ray diffraction (XRD) patterns; and (g) stress-strain curves of GC products pyrolyzed at 800 and 1000° C., respectively.

FIG. 11 depicts (a) SEM images of as-printed honeycomb structure using PN-V-1 resin; and (b) SEM images and elemental mapping of the pyrolysed honeycomb structure.

FIG. 12 depicts (a) T_{g} and carbon yield of PN-T-1 and PN-V-1 compared with literature results (Halim, S. I. A. et al., Macromol. Symp., 2016, 365, 95; Wang, Y. et al., Progress in Organic Coatings, 2015, 78, 404; Maity, T. et al., Materials Science and Engineering A, 2007, 464, 38; Wang, C. & Shieh, J., Journal of Applied Polymer Science, 1999, 73, 353; Liu, Q. et al., Polymer-Plastics Technology and Engineering, 2012, 51, 251; Vahabi, H. et al., Progress in Organic Coatings, 2018, 123, 160; Yu, H. et al., Polymer, 2011, 52, 4891; Joseph, P. & Tretsiakova-McNally, S., Polymer Degradation and Stability, 2012, 97, 2531; Zhang, S. et al., Composites Science and Technology, 2017, 152, 165; Huang J. et al., Polymer, 2003, 44, 4491; Amarnath, N. et al., ACS Sustainable Chem. Eng., 2018, 6, 15151; Weigand, J. J. et al., Polymer, 2020, 189, 122193; Hegde, M. et al., Adv. Mater., 2017, 1701240); (b) shrinkage and carbon yield of printed carbons from reported precursors and PN-T-1 and PN-V-1 (PI: Arrington, C. B. et al., ACS Macro Lett., 2021, 10, 412; PAN & PBZ-C5: Jacoben, A. J. et al., Carbon, 2011, 49, 1025; Phenolic: Szczurek, A. et al., Carbon, 2015, 88, 70; PBZ-C2: Lu, Y., Chem. Commun., 2021, 57, 3375; commercial acrylate resins: Rezaei, B. et al., Materials and Design, 2020, 193, 108834; Wang, P. et al., Adv. Mater. Technol., 2020, 5, 1901030; Narita, K. et al., Adv. Energy Mater., 2021, 11, 2002637). (*commercial resin data from our test).

### DESCRIPTION

As mentioned hereinbefore, it has been surprisingly found that certain phthalonitrile (PN) compounds have unique properties that make them particularly suited to additive manufacture and the production of GC with a complex (e.g. 3D) structure. Thus, in a first aspect of the invention, there is provided a compound of formula I:

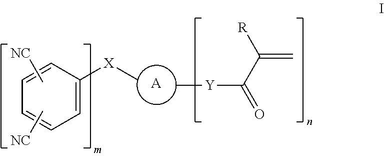

wherein:
m and n are independently from 1 to 3;
X is a bond, O or NH;
R is H or an aliphatic group; and
A is an aromatic, a heterocyclic or an aliphatic group and is unsubstituted or is substituted by one of more of the group consisting of halo, OR^{1a}, and NR^{1b}R^{1c};
R^{1a} to R^{1c} are each independently selected from H and C₁ to C₁₀ alkyl; and
Y is a bond, a C₁ to C₁₀ alkylene group or a —C₁ to C₁₀ alkylene-O— group, where in the latter group the alkylene portion is directly attached to A and the oxygen atom is directly attached to the carbon of the C═O double bond.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a composition” includes mixtures of two or more such compositions, and the like.

Compounds of formula I may contain double bonds and may thus exist as E (entgegen) and Z (zusammen) geometric isomers about each individual double bond. All such isomers and mixtures thereof are included within the scope of the invention.

Compounds of formula I may exist as regioisomers and may also exhibit tautomerism. All tautomeric forms and mixtures thereof are included within the scope of the invention.

Compounds of formula I may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise states, the term “aliphatic” may refer to an alkyl group. Unless otherwise stated, the term “alkyl” refers to an unbranched or branched, acyclic or cyclic, saturated or unsaturated (so forming, for example, an alkenyl or alkynyl) hydrocarbyl radical, which may be substituted or unsubstituted (with, for example, one or more halo atoms). Where the term “alkyl” and “aliphatic” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” and “aliphatic” is a cyclic group (which may be where the group “cycloalkyl” is specified), it is preferably C₃₋₁₂ cycloalkyl and, more preferably, C₅₋₁₀ (e.g. C₅₋₇) cycloalkyl.

Unless otherwise specified herein, the term “heterocyclic” may be 4- to 14-membered, such as a 5- to 10-membered (e.g. 6- to 10-membered), heterocyclic group that may be aromatic, fully saturated or partially unsaturated, and which contains one or more heteroatoms selected from O, S and N, which heterocyclic group may comprise one or two rings. Examples of hetereocyclic groups that may be mentioned herein include, but are not limited to azetidinyl, dihydrofuranyl (e.g. 2,3-dihydrofuranyl, 2,5-dihydrofuranyl), dihydropyranyl (e.g. 3,4-dihydropyranyl, 3,6-dihydropyranyl), 4,5-dihydro-1H-maleimido, dioxanyl, dioxolanyl, furanyl, furazanyl, hexahydropyrimidinyl, hydantoinyl, imidazolyl, isothiaziolyl, isoxazolidinyl, isoxazolyl, morpholinyl, 1,2- or 1,3-oxazinanyl, oxazolidinyl, oxazolyl, piperidinyl, piperazinyl, pyranyl, pyrazinyl, pyridazinyl, pyrazolyl, pyridinyl, pyrimidinyl, pyrrolinyl (e.g. 3-pyrrolinyl), pyrrolyl, pyrrolidinyl, pyrrolidinonyl, 3-sulfolenyl, sulfolanyl, tetrahydrofuranyl, tetrahydropyranyl, tetrahydropyridinyl (e.g. 3,4,5,6-tetrahydropyridinyl), 1,2,3,4-tetrahydropyrimidinyl, 3,4,5,6-tetrahydropyrimidinyl, tetrahydrothiophenyl, tetramethylenesulfoxide, tetrazolyl, thiadiazolyl, thiazolyl, thiazolidinyl, thienyl, thiophenethyl, triazolyl and triazinanyl.

In embodiments of the invention, there the heterocyclic group is aromatic, it may be referred to as a heteroaryl group. Heteroaryl groups that may be mentioned include benzothiadiazolyl (including 2,1,3-benzothiadiazolyl), isothiochromanyl and, more preferably, acridinyl, benzimidazolyl, benzodioxanyl, benzodioxepinyl, benzodioxolyl (including 1,3-benzodioxolyl), benzofuranyl, benzofurazanyl, benzothiazolyl, benzoxadiazolyl (including 2,1,3-benzoxadiazolyl), benzoxazinyl (including 3,4-dihydro-2H-1,4-benzoxazinyl), benzoxazolyl, benzomorpholinyl, benzoselenadiazolyl (including 2,1,3-benzoselenadiazolyl), benzothienyl, carbazolyl, chromanyl, cinnolinyl, furanyl, imidazolyl, imidazo[1,2-a]pyridyl, indazolyl, indolinyl, indolyl, isobenzofuranyl, isochromanyl, isoindolinyl, isoindolyl, isoquinolinyl, isothiaziolyl, isoxazolyl, naphthyridinyl (including 1,6-naphthyridinyl or, preferably, 1,5-naphthyridinyl and 1,8-naphthyridinyl), oxadiazolyl (including 1,2,3-oxadiazolyl, 1,2,4-oxadiazolyl and 1,3,4-oxadiazolyl), oxazolyl, phenazinyl, phenothiazinyl, phthalazinyl, pteridinyl, purinyl, pyranyl, pyrazinyl, pyrazolyl, pyridazinyl, pyridyl, pyrimidinyl, pyrrolyl, quinazolinyl, quinolinyl, quinolizinyl, quinoxalinyl, tetrahydroisoquinolinyl (including 1,2,3,4-tetrahydroisoquinolinyl and 5,6,7,8-tetrahydroisoquinolinyl), tetrahydroquinolinyl (including 1,2,3,4-tetrahydroquinolinyl and 5,6,7,8-tetrahydroquinolinyl), tetrazolyl, thiadiazolyl (including 1,2,3-thiadiazolyl, 1,2,4-thiadiazolyl and 1,3,4-thiadiazolyl), thiazolyl, thiochromanyl, thiophenetyl, thienyl, triazolyl (including 1,2,3-triazolyl, 1,2,4-triazolyl and 1,3,4-triazolyl) and the like. Substituents on heteroaryl groups may, where appropriate, be located on any atom in the ring system including a heteroatom. The point of attachment of heteroaryl groups may be via any atom in the ring system including (where appropriate) a heteroatom (such as a nitrogen atom), or an atom on any fused carbocyclic ring that may be present as part of the ring system. Heteroaryl groups may also be in the N- or S-oxidised form. Particularly preferred heteroaryl groups include pyridyl, pyrrolyl, quinolinyl, furanyl, thienyl, oxadiazolyl, thiadiazolyl, thiazolyl, oxazolyl, pyrazolyl, triazolyl, tetrazolyl, isoxazolyl, isothiazolyl, imidazolyl, pyrimidinyl, indolyl, pyrazinyl, indazolyl, pyrimidinyl, thiophenetyl, thiophenyl, pyranyl, carbazolyl, acridinyl, quinolinyl, benzoimidazolyl, benzthiazolyl, purinyl, cinnolinyl and pterdinyl. Particularly preferred heteroaryl groups include monocylic heteroaryl groups.

Unless otherwise specified herein, a “carbocyclic ring system” may be 4- to 14-membered, such as a 5- to 10-membered (e.g. 6- to 10-membered, such as a 6-membered or 10-membered), carbocyclic group that may be aromatic, fully saturated or partially unsaturated, which carbocyclic group may comprise one or two rings. Examples of carbocyclic ring systems that may be mentioned herein include, but are not limited to cyclobutyl, cyclopentyl, cyclohexyl, cyclooctyl, phenyl, naphthyl, decalinyl, tetralinyl, bicyclo[4.2.0]octanyl, and 2,3,3a,4,5,6,7,7a-octahydro-1H-indanyl. Particularly preferred carbocyclic groups include phenyl, cyclohexyl and naphthyl.

It will be appreciated that the disclosure herein allows for a range of PN materials that can be used in the production of suitable complex 3D GC structures. As such, it is believed that a range of different PN materials having the essential structural requirements of the compound of formula I may be suitable for use in the formation of resins and hence the complex 3D GC structures. More particular compounds of formula I that may be mentioned herein may be those where one or more of the following apply:
(i) A may be an aromatic or aliphatic group and is unsubstituted or is substituted by one of more of the group consisting of OR^{1a}, and NR^{1b}R^{1c} (e.g. A may be phenyl and is unsubstituted or is substituted by one of more of the group consisting of OR^{1a});
(ii) R^{1a} to R^{1c}, where present, may be H or CHs;
(iii) X may be NH or O, optionally wherein X may be O;
(iv) n and m may both be 1;
(v) Y may be a —C₁ to C₃ alkylene-O— group, where the alkylene portion is directly attached to A and the oxygen atom is directly attached to the carbon of the C═O double bond;
(vi) R may be H or CHs, optionally wherein R may be H.

In particular embodiments of the invention disclosed herein, the compound of formula I may be selected from:

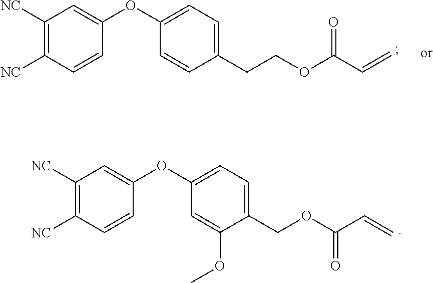 [SMILES: C=CC(=O)OCCc1ccc(Oc2ccc(C#N)c(C#N)c2)cc1 ; C=CC(=O)OCc1ccc(Oc2ccc(C#N)c(C#N)c2)cc1OC]

As will be appreciated, the combination of two or more PNs according to the compound of formula I may be used herein (e.g. in resin precursor formulations, as discussed below). As such, the combination of the two PNs above is contemplated.

As will be appreciated, the monomeric structured disclosed hereinbefore are particularly suitable for the formation of complex 3D structures and hence can also provide complex 3D GC structures. As such, in a further aspect of the invention, there is provided resin precursor formulation for additive manufacturing, comprising:
a compound of formula I as defined hereinbefore;
a photoinitiator; and
an organic solvent suitable for use in additive manufacturing.

The resin precursor formulation disclosed above may be used as-is, or it may further contain a crosslinking agent if that is desired or required, plus other additives. Thus, in embodiments of the invention, the formulation may further comprise one or more of a crosslinking agent, a photoabsorber and a filler.

Any suitable crosslinking agent may be used herein. For example, the crosslinking agent may be one suitable for use with acrylate groups, such crosslinkers may include a diacrylate or a polyacrylate (e.g. from 2 to 5 acrylate groups). In particular embodiments of the invention the crosslinking agent may be bisphenol A ethoxylate diacrylate. In embodiments of the invention that may be mentioned herein, a crosslinker may be present. For example, if the compound of formula I is not able to crosslink by itself, the presence of a crosslinking agent may be desired to ensure that a resulting 3D printed resin structure has suitable storage modulus and Tg, thereby ensuring that the 3D printed structure is a high-performance material with excellent thermal and mechanical properties.

Any suitable photoabsorber may be used herein when desired. For example, the photoabsorber may be Sudan I. Adding a photoabsorber (which absorbs at the wavelength of a laser used in additive manufacture) to the resin may allow for the absorption of stray and scattered light, thus sustaining the spot size of the laser. According to the printing resolution requirement or the printing limitations of a printer, the addition of a photoabsorber can adjust the curing depth of the resin during printing to enable a successful printing work.

Any suitable filler may be used in the resin precursor formulations disclosed herein. For example, the filler may be an organic filler and/or an inorganic filler. Organic or inorganic fillers can be added into the resin formulations, which may result in the tuning and functionalizing of the final GC products based on the requirement for said product.

The formulation may further comprise one or more modifying agents as desired and within the scope of polymer and additive manufacture formulations. These modifying agents may be used to ensure that the final GC product has certain desirable properties and/or structures.

Any suitable photoinitiator may be used in the resin precursor formulations disclosed herein. Examples of suitable photoinitiators include, but are not limited to 2,4,6-trimethyl benzoyldiphenyl phosphine oxide, 1-hydroxycyclohexyl phenyl ketone, 2-hydroxy-2-methylphenyl-propane-1-one, phenylbis(2,4,6-trimethylbenzoyl)phosphine oxide), and combinations thereof. For example the photoinitiator may be phenylbis(2,4,6-trimethylbenzoyl)phosphine oxide).

Any suitable organic solvent may be used in the resin precursor formulations disclosed herein. That is any organic solvent that can be used in the resin formulation disclosed herein may be used, provided that it is compatible with the other components of the formulation. Examples of suitable organic solvents include, but are not limited to toluene, chloroform, tetrahydrofuran, and more particularly, toluene, dimethylformamide, and N-methylpyrrolidine For example the organic solvent may be N-methylpyrrolidine.

In particular embodiments of the invention, the resin precursor formulation may be one that comprises:
a compound of formula I as defined in any one of Claims 1 to 9;
a photoinitiator;
an organic solvent suitable for use in additive manufacturing;
a crosslinking agent; and
a photoabsorber.

In yet more particular embodiments of the invention, the resin precursor formulation may comprises:
a compound of formula I as defined in any one of Claims 1 to 9;
phenylbis(2,4,6-trimethylbenzoyl)phosphine oxide);
N-methylpyrrolidine;
bisphenol A ethoxylate diacrylate; and
Sudan I.

The resin precursor formulation disclosed herein may be used in any suitable additive manufacturing process. For the avoidance of doubt, the terms “additive manufacture” and “3D printing” may be used herein interchangeably. In particular embodiments of the invention that may be mentioned herein the resin precursor formulations disclosed herein may be suitable for use in a stereolithography-type additive manufacturing process, such as stereolithography and/or digital light processing.

As will be appreciated, once the resin undergoes an additive manufacturing process, it will provide a resin. As such, in a further aspect of the invention, there is provided a polymeric resin composition obtained from the polymerisation of a compound of formula I as described hereinbefore.

As noted hereinbefore, the resin provided by the resin precursor formulations and hence the compounds of formula I disclosed herein may have superior properties. For the example, the polymeric resin compositions of the current invention has one or more of the following properties:
(a) a T_{d} that is greater than or equal to 400° C.;
(b) a T_{g} that is greater than or equal to 300° C.;
(c) a bending strength that is greater than or equal to 100 MPa, such as greater than or equal to 150 MPa;
(d) a modulus greater than or equal to 3.5 Gpa;
(e) a carbon yield, following a sintering process, of greater than or equal to 60%; and
(f) a shrinkage value of from 20 to 40%, such as from 25 to 30%, for a glassy carbon three-dimensional object relative to a green polymeric resin three-dimensional product following a sintering process to provide the glassy carbon three-dimensional product.

As will be appreciated, the resin precursor formulations disclosed herein may be suitable for use in additive manufacture and so the polymeric resin produced may be formed as a three-dimensional object.

As will be appreciated, the resin precursor formulation is suitable for use in an additive manufacturing process. Thus, in a further aspect of the invention, there is provided a process of additive manufacturing, the process comprising:
(ai) providing a resin precursor formulation for additive manufacturing as described hereinbefore; and
(aii) controlling a stereolithography-type apparatus to form a three-dimensional object by using the resin precursor formulation, wherein the resin precursor formulation is deposited by the apparatus on a surface in a layer by layer operation and each layer is subjected to a polymerisation reaction before each subsequent layer is laid down. Further details of how the additive manufacturing may be conducted are provided in the examples section below.

As noted above, the resin compositions disclosed hereinbefore may be particularly suited to the formation of 3D glassy carbon objects. Thus, in yet a further aspect of the invention, there is provided a process of providing a three-dimensional glassy carbon object, the process comprising the steps of:
(bi) providing a three-dimensional object comprising a polymeric resin material obtained from the polymerisation of a compound of formula I as described herein; and
(bii) subjecting the three-dimensional object to carbonisation through the application of heat to provide a glassy carbon object. Further details of how the formation of 3D glassy carbon objects may be conducted are provided in the examples section below.

In yet further aspect of the invention there is also provided:
(aa) use of a compound of formula I as described herein in a method of additive manufacture; and
(ab) use of a compound of formula I as described herein in a method providing a glassy carbon object.

For the avoidance of doubt, the term 3D as used herein is intended to refer to an object that has a three-dimensional structure. Based on the printer used, various three dimensional structures can be printed with the resins

This invention relates to
(1) photocurable and 3D printable PN (3DPN), their derivatives and formulated resins;
(2) their precision 3D printing; and
(3) their conversion to various carbonized structure such as GC with ultralow shrinkage.

As described in more detail by the examples, the PN-based resins disclosed herein are high-performance resins with excellent thermal and mechanical properties. Moreover, the resins are promising carbon precursors for high-resolution 3D printing of GC products for high temperature applications. Compared to reported printable carbon precursor resins, these 3DPN based resins display a great deal of variety, adaptability and functionality, which can provide ultra-high carbon yield and precise complex 3D structures of GC products.

Particular advantages that may apply to one or more of the embodiments of the invention include:
[1] The 3DPN monomers have good solubility in common organic solvents, such as toluene, dimethylformamide (DMF), N-methylpyrrolidone (NMP), etc. Therefore, various additives, e.g., cross-linkers fillers, and modifiers, can be used with good compatibility.
[2] The 3DPN resins can be photocured at room temperature and be post processed in temperature range of from about 200 to 300° C.
[3] The 3DPN resins are high-performance resins with thermal degradation temperature (T_{d})>400° C., T_{g}˜300° C., bending strength in the order of 150 MPa, Modulus higher than 3.5 GPa.
[4] The 3DPN resins have high carbon yield, >60% which is significantly higher than any reported 3D printable resins.
[5] SLA (Stereolithography) type 3D printing technology such as SLA and DLP (Digital Light Processing) can be applied for the resins.
[6] GC objects with complex 3D structures can be obtained with structural integrity and fidelity.
[7] An ultralow shrinkage of from about 25 to 30% of GC products compared to printed green body can be obtained.
[8] Organic or inorganic fillers can be added into the resins, resulting in the tuning and functionalizing of the final GC products based on demands.
[9] Possibility of conversion to other forms of 3D printed carbon structures is also possible by varying the heat treatment temperature and process.
   There are numerous applications of high-performance resins and glassy carbon materials both in laboratory research and industries. For example, the following materials, technology and processing are desirable for using glassy carbon materials. 3DPN based resins for heat-resistance coatings.
   3DPN based resins for high-performance composites and blends to be applied under harsh environments.
   3DPN based resins as potential flame retardant materials.
   GC based electrochemical devices.
   Sensors, Electrodes (e.g., energy storage), Bipolar plates.
   GC based biomedical devices.
   Implants, tissue engineering and drug delivery devices.
   Biosensors, e.g., carbon microarray.
   GC based tools for precision molding/micro-molding process for glass etc.
   GC based conducting and high temperature microfluidics.
   GC based electromagnetic interference shielding devices.
   GC based lightweight conducting materials (e.g., space technology).

Further applications of the invention may also include used in medical implants, for example to produce printed non-metallic glass carbon screws.

Further aspects and embodiments of the invention will now be described by reference to the following non-limiting examples.

### EXAMPLES

### Reading Keys:

Blue—2022-327 Write Up_Final.docx
Brown—2022-327_Additional Information.docx

The disclosure relates to develop 3D printable high performance phthalonitrile (3DPN) based polymers as well as 3D printing of glassy carbon with ultra-high carbon yield and low shrinkage using the developed PN based resins via high-resolution SLA type additive manufacture technology.

### Materials

4-Nitrophthalonitrile, anhydrous potassium carbonate, anhydrous dimethyl sulfoxide (DMSO), bisphenol A ethoxylate diacrylate (BPAEDA, Mₙ˜468), phenylbis(2,4,6-trimethylbenzoyl) phosphine (BAPO), pyrogallol, and Sudan I were purchased from Sigma Aldrich. Tyrosol (2-(4-hydroxyphenyl)ethanol), vanillyl alcohol (4-hydroxy-3-methoxybenzyl alcohol) and acryloyl chloride were purchased from Tokyo Chemical Industry. All chemicals were of reagent grade and used as received unless stated otherwise.

### Example 1. General Preparation of 3D Printable PN (3DPN) Monomers

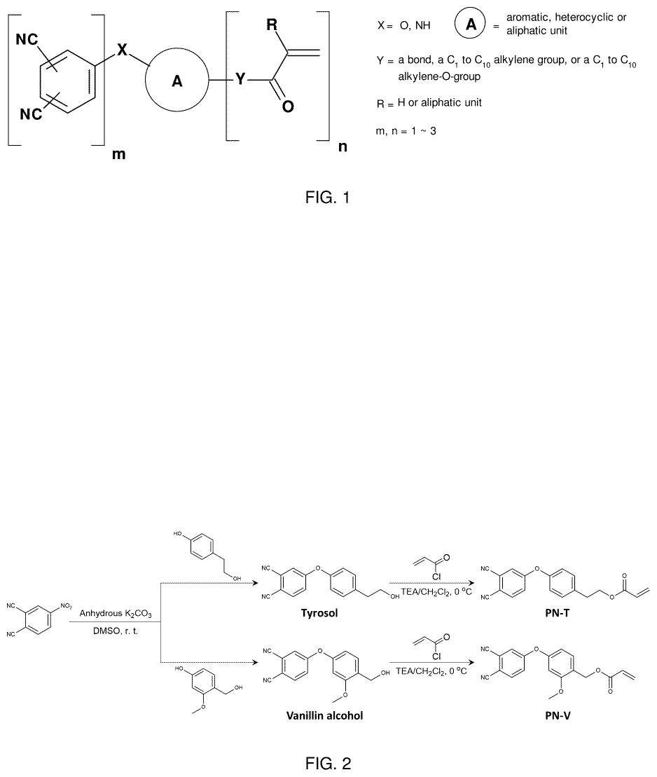

The PN type monomers are generally functionalized with photocurable groups. The generic structured PN type monomers are presented in FIG. 1, which are including PN type moiety, linker (aromatic or aliphatic unit) and photocurable functional group(s).

The synthetic approach to the 3DPN monomers is as the following. Firstly, nitro-PN was reacted with hydroxyl substituted phenol to synthesize the hydroxyl-terminated PNs. Hydroxyl-phenol, nitro-PN and K₂CO₃ were reacted in DMSO at room temperature for 48 h. Afterwards, the reaction mixture was poured into water and collected. The as-prepared hydroxyl-PN precursors were continuously reacted with acryloyl chloride to form the acrylate-functionalized PN monomers and their chemical structures were verified by ¹H Nuclear Magnetic Resonance (NMR) Spectrometry and Fourier Transform Infrared (FT-IR) Spectroscopy respectively.

### Preparation of Hydroxyl-PN Precursors

Phenolic based alcohol, PN type compound and base with molar ratio of 1:1:2.3 were added into three-neck round bottom flask and mechanically stirred at controlled temperature for a period in DMSO solvent under nitrogen (N₂) atmosphere. At the end of this time the reaction mixture was poured into a large scale of ice-water, filtered off and washed with plenty of water. The crude product was collected and dried under vacuum to get the hydroxyl-PN precursors for next step usage.

### Preparation of 3DPN Monomers

To an ice-bath cooled anhydrous dichloromethane (CH₂Cl₂) solution with previously prepared hydroxyl-PN precursor (1 eq.) and base (1.3 eq.), the acryloyl chloride (or acryloyl chloride derivatives) was added in slowly under N₂ atmosphere. Upon the complete addition, the reaction solution was brought to room temperature and continuously stirred for some time until the completion of the reaction. Then the formed precipitate was filtered off and the CH₂Cl₂ was removed under vacuum. The residue was dissolved in ethyl acetate and filtered again. The obtained ethyl acetate solution was washed 3 times with basic solution and then once with deionised (DI) water. The organic layer was collected, dried over anhydrous sodium sulphate, filtered and evaporated. The crude product was purified by crystallisation with the mixture of hexane and ethyl acetate (1:1) to get the desired 3DPN monomer as a white solid.

### ¹H Nuclear Magnetic Resonance (NMR) Spectroscopy

1H spectra were measured by a JOEL ECA400 nuclear magnetic resonance (NMR) spectrometer. Deuterated chloroform (CDCls₃) was used as solvent and tetramethylsilane was used as internal standard.

### Attenuated Total-Reflectance Fourier Transform Infrared (ATR-FTIR) Spectroscopy and Fourier Transform Infrared (FT-IR) Spectroscopy

Attenuated total-reflectance Fourier transform infrared (ATR-FTIR) spectra and Fourier transform infrared (FT-IR) spectra were recorded by Perkin Elmer Frontier FTNIR/MIR spectrometers.

### Example 2. Preparation of 4-[4-(2-acrylicethyl)phenoxy]phthalonitrile (PN-T) and 4-[3-methoxyl-4-acrylicmethyl)phenoxy]phthalonitrile (PN-V)

PN-T and PN-V were prepared as described in the current example by following the general protocol described in Example 1.

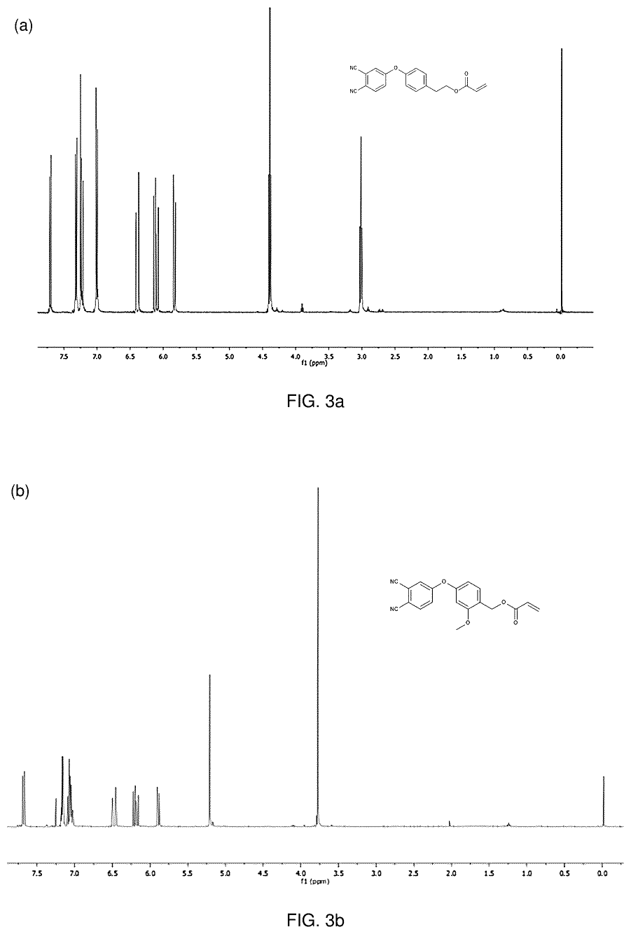

With the aim at obtaining PN monomers endowed with photopolymerisable capability, monoacrylate functionalized PN compounds, namely PN-T and PN-V, were prepared starting from 4-nitrophthalonitrile via the synthetic approach shown in FIG. 2. Firstly, coupling reaction was employed to prepare a hydroxyl-terminated PN under alkaline condition in solvent of DMSO. Being reacted at room temperature for 48 h, the reacting solution was poured into a great scale of water, leading to the formation of a large amount of precipitates, which were washed thoroughly with plenty of water, collected by suction filtration and dried under vacuum to obtain the desired products. The as-prepared hydroxyl-PN precursor was then reacted with acryloyl chloride to obtain the crude acrylate-functionalized PN product, which was subsequently purified by recrystallisation to get the final products PN-T and PN-V. Their chemical structures were verified by ¹H NMR and FT-IR resonances respectively.

### Preparation of 4-[4-(2-acrylicethyl)phenoxy]phthalonitrile (PN-T)

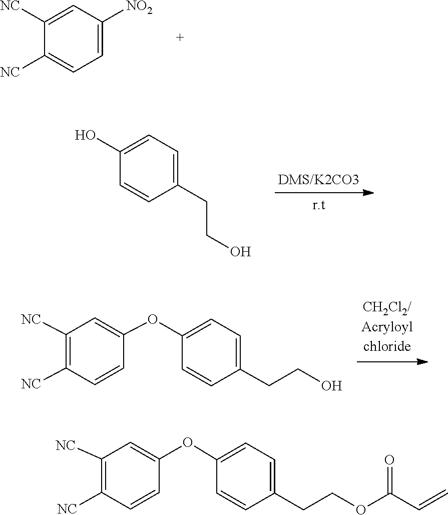 [SMILES: N#Cc1ccc([N+](=O)[O-])cc1C#N ; OCCc1ccc(O)cc1 ; N#Cc1ccc(Oc2ccc(CCO)cc2)cc1C#N ; C=CC(=O)OCCc1ccc(Oc2ccc(C#N)c(C#N)c2)cc1]

2-(4-Hydroxyphenyl)ethanol (13.8 g) and K₂CO₃ (20.7 g) were added to a solution of 4-nitrophthalonitrile (17.3 g) compound under stirring in dimethyl sulfoxide (DMSO) (100 mL). The mixture was allowed at room temperature for 48 h, poured into water and extracted with dichloromethane. The extracts were combined and dried over sodium sulphate, then filtered and the solvent of the filtrate was evaporated. The residue subsequently reacted with acryloyl chloride and anhydrous triethylamine in an ice-bath cooled CH₂Cl₂ solution. The solution was brought to room temperature and continuously stirred for 3 h to complete the reaction. Then the CH₂Cl₂ solution was washed with saturated NaHCO₃ solution and DI water. The CH₂Cl₂ layer was collected, dried, filtered and CH₂Cl₂ was removed under vacuum. The crude product was purified by crystallisation with the mixture of hexane and ethyl acetate (1:1) to get the desired PN monomer as a white solid. (White solid, yield: 62%). ¹H NMR (400 MHz, CDCl₃): δ (ppm) 7.72 (dd, 1H), 7.32 (dd, 2H), 7.24-7.21 (m, 2H), 7.00 (dd, 2H), 6.39 (dd, 1H), 6.11 (q, 1H), 5.83 (dd, 1H), 4.39 (t, 2H), 3.02 (t, 2H). FT-IR (KBr, cm⁻¹): 3116-3009 (Ar—CH), 2981-2830 (aliphatic-CH), 2233 (Ar—CN), 1721 (—C═O—), 1635 (—C═C—), 1256 (Ar—O—Ar).

### Preparation of 4-[3-methoxyl-4-acrylicmethyl)phenoxy]phthalonitrile (PN-V)

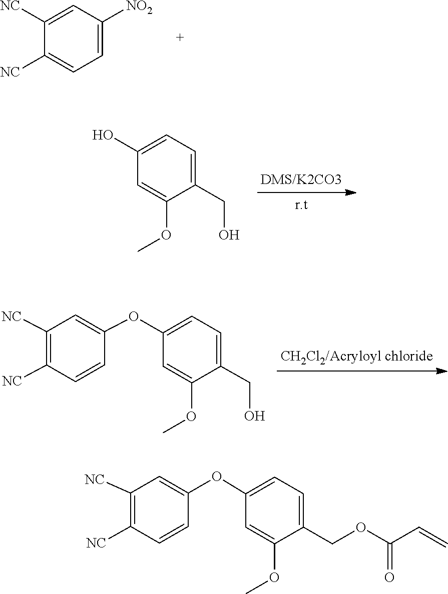

4-Hydroxy-3-methoxybenzyl alcohol (15.4 g) and K₂CO₃ (20.7 g) were added to a solution of 4-nitrophthalonitrile (17.3 g) compound under stirring in dimethyl sulfoxide (DMSO) (100 mL). The mixture was allowed at room temperature for 48 h, poured into water and extracted with dichloromethane. The extracts were combined and dried over sodium sulphate, then filtered and the solvent of the filtrate was evaporated. The residue subsequently reacted with acryloyl chloride and anhydrous triethylamine in an ice-bath cooled CH₂Cl₂ solution. The solution was brought to room temperature and continuously stirred for 3 h to complete the reaction. Then the CH₂Cl₂ solution was washed with saturated NaHCO₃ solution and DI water. The CH₂Cl₂ layer was collected, dried, filtered and CH₂Cl₂ was removed under vacuum. The crude product was purified by crystallisation with the mixture of hexane and ethyl acetate (1:1) to get the desired PN monomer as a white solid. (White solid, yield: 56%). ¹H NMR (400 MHz, CDCl₃): δ (ppm) 7.68 (m, 1H), 7.18-7.15 (m, 2H), 7.10-7.04 (m, 3H), 6.48 (dd, 1H), 6.19 (q, 1H), 5.89 (dd, 1H), 5.21 (s, 2H), 3.77 (s, 3H). FT-IR (KBr, cm⁻¹): 3149-3026 (Ar—CH), 2993-2824 (aliphatic-CH), 2233 (Ar—CN), 1727 (—C═O—), 1634 (—C═C—), 1251 (Ar—O—Ar).

### Results and Discussions

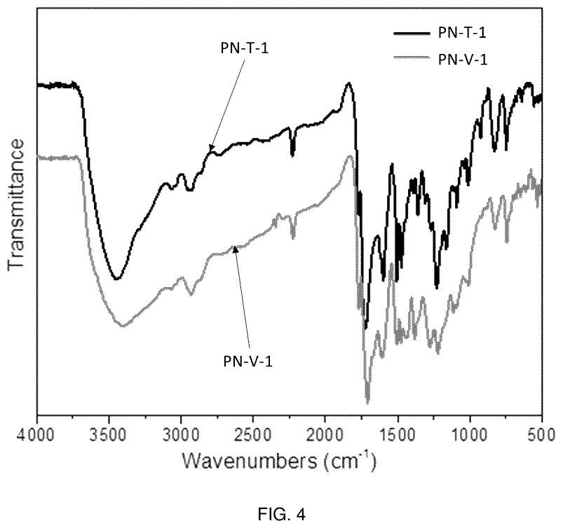

As shown in FIG. 3, the characteristic proton resonances of the acrylate group (—CH═CH₂) appears at 6.39, 6.11, 5.83 ppm for PN-T, and at 6.48, 6.19, 5.89 ppm for PN-V, thus indicating the acrylation of the hydroxyl-PN compounds. Additionally, FT-IR absorption spectra (FIG. 4) clearly exhibit the characteristic absorption band of —C≡N at ˜2233 cm⁻¹ and the stretching absorption of carbonyl and vinyl groups of acrylate structure at ˜1721 and ˜1635 cm⁻¹, respectively, further suggesting the successful preparation of the acrylated-PNs.

### Example 3. General Formulation of 3DPN-Based Carbon Precursor Resins

Photopolymerizable resins based on the prepared 3DPN monomer, a commercial diacrylate and other additives can be formulated.

3DPN monomer was completely dissolved in organic solvents. Then BAPO and crossl-inker were added in. The resulted solution was homogenised with a vortex mixer for 30 s and subsequently allowed to stand at room temperature for 2 h to ensure the absence of bubbles.

### Example 4. Resin Formulations Based on PN-T or PN-V Monomers

### Preparation of PN-T-1 and PN-V-1 Resins

PN-T (or PN-V) was firstly dissolved in dimethylformamide (DMF) to form a solution with a concentration of 1.125 g/mL. Then, 5 wt % of BPAEDA (cross-linker) was added to the solution and homogenized with a vortex mixer for 30 s. 2 wt % BAPO (photoinitiator), Sudan I (0.05 wt %) (photoabsorber) and pyrogallol (0.01 wt %) (free radical stabilizer) were subsequently weighed, added in and vigorously mixed. The resultant mixture was then allowed to stand at room temperature for 2 h to ensure the absence of bubbles. The homogeneous resins formed were named PN-T-1 and PN-V-1 respectively.

### Example 5. Structural Characterisation, Thermal Stability and Mechanical Properties

Various samples were fabricated by firstly set in silicone moulds, followed by exposure to ultraviolet (UV) irradiation. They were then proceeded to post treatment for solvent removal and thermal curing. Eventually, thermally cured PN-T-1 and PN-V-1 were obtained and evaluated for structural, thermal and mechanical properties.

### Sample Preparation by Photo and Thermal Cure

Samples for FT-IR, Differential Scanning Calorimetry (DSC), TGA, DMA and 3-point bending test were prepared by UV photocuring uniform samples firstly, followed by thermal curing with a progressive heat treatment. Typically, resin PN-T-1 or resin PN-V-1 without BAPO and Sudan I was added into silicone moulds and photocured in a UV chamber for 3 min. The specimens were demoulded, flipped over and photocured in the UV chamber for another 3 min. The photocured samples were then transferred to a vacuum oven to remove most of the solvent under vacuum at 50° C. for 24 h. The resultant samples were subjected to further thermal treatment with the following heating schedule: 120° C. (1 h), 160° C. (1 h), 180° C. (1 h), 200° C. (1 h), 220° C. (1 h), 240° C. (1 h) and 260° C. (12 h).

### Differential Scanning Calorimetry (DSC)

Differential scanning calorimetry (DSC) (TA Instruments 2010) was performed from room temperature to 300° C. at a constant heating rate of 10° C./min under nitrogen atmosphere.

### Thermogravimetric Analysis (TGA)

Thermogravimetric analysis (TGA) measurements were performed on TA Instruments 2950 under a nitrogen atmosphere at a heating rate of 10° C./min.

### Measurement of the Flexural Properties by Three-point Bending Test

The flexural properties were measured by three-point bending tests with loading speed 1 mm/min using a mechanical tester Instron 5567. The cross-head speed is fixed at 0.1 mm/min for all samples.

### Dynamic Mechanical Analysis (DMA)

Dynamic mechanical analysis (DMA) was carried out with TA instruments Q800 DMA utilizing the single cantilever mode with temperature ramp from ambient to 300° C.

### Results and Discussions

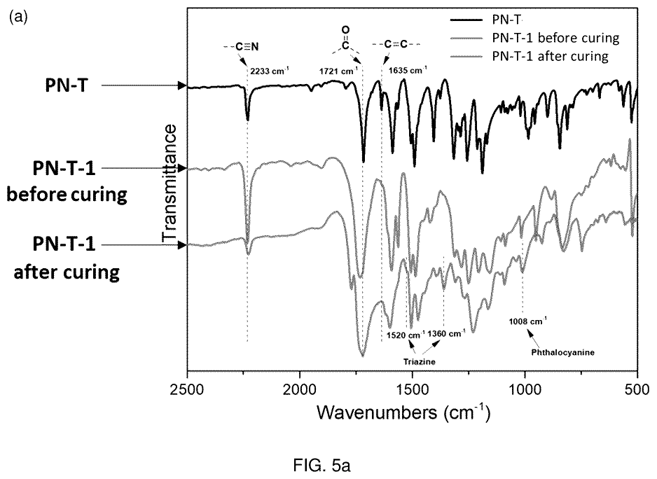

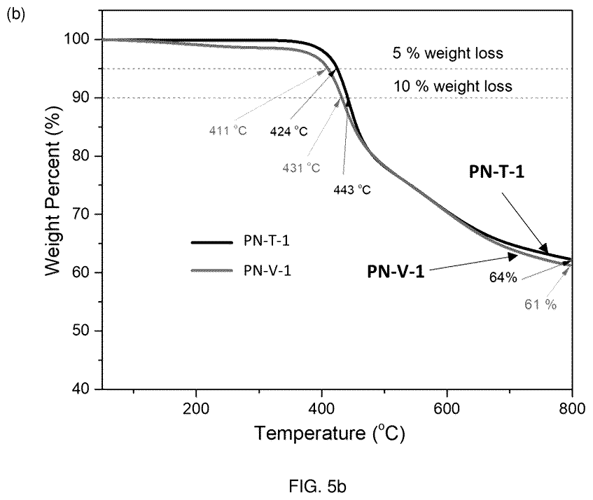

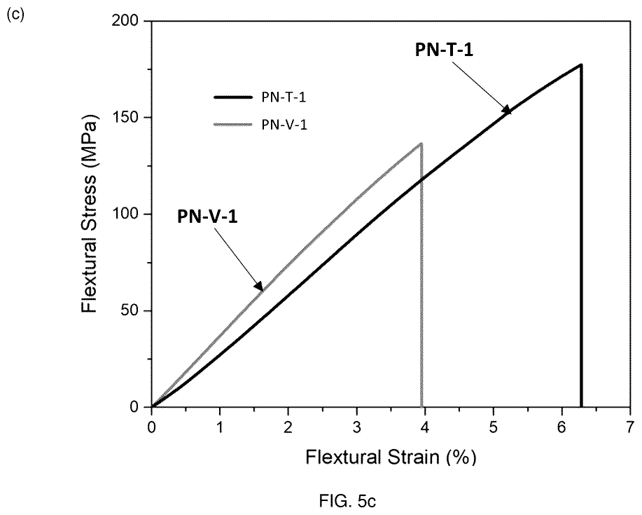

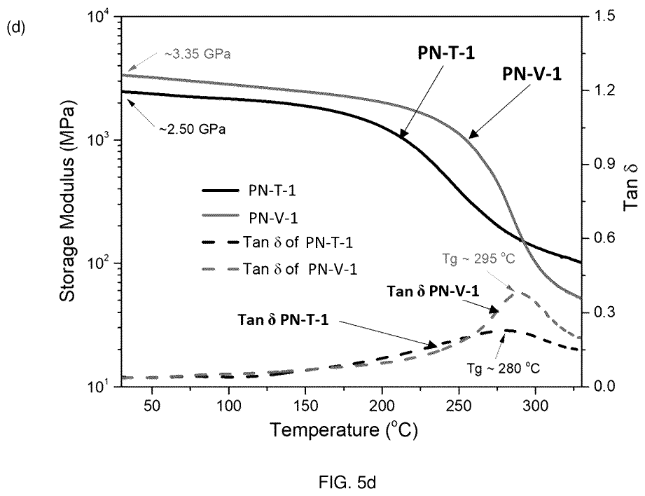

FT-IR spectroscopy was conducted to investigate structural changes of the PN resins before and after post-treatment. As shown in FIG. 5a, the FT-IR characterisation of PN-T-1 is representative. Compared to the PN-T monomer compound, which clearly exhibits a band of double bond at ˜1637 cm⁻¹, PN-T-1 does not show any IR peak at the same position, indicating the complete consumption of double bonds in the resin. The characteristic peak of nitrile group is clearly observed at ˜2233 cm⁻¹ for both PN-T and PN-T-1, while after being thermally treated with progressive temperatures, the peak intensity decreases dramatically due to the cyclisation of nitrile groups into triazine and phthalocyanine structures under the alleviated temperature. As a result, the typical IR peaks at 1520 and 1360 cm⁻¹ for triazine and the peak at 1008 cm⁻¹ for phthalocyanine are present in the spectrum of the thermally treated PN-T-1 product. It is also worth noting that there is an obvious IR band of OH group appearing (FIG. 4) after the thermal treatment, suggesting the formation of carboxylic acid within polymer network. This may be attributed to the partial degradation of acrylate segments under long-term thermal treatment. However, it is known that acid compounds can catalyse the cyclisation of nitrile groups (Jia, Y. et al., Polymers, 2022, 14, 219), resulting in a lower thermal curing temperature of the product.

Next, thermal properties of the resins were examined using TGA analysis under nitrogen atmosphere. FIG. 5b shows the TGA thermograms of PN-T-1 and PN-V-1 before and after thermal curing respectively. It is found that the weight loss of 5 wt % (T₅) occurred at 424 and 411° C. for thermally cured PN-T-1 and PN-V-1, indicating that their thermal degradation temperature (T_{d}) is over 400° C. The significant thermal stability is believed to be the attribution of the highly cross-linked PN units among the polymer chains. More interestingly, when without a thermal heat treatment prior to carbonisation, the char yield of the PN-T-1 and PN-V-1 resins is from 55 to 58% at 800° C. After thermal curing, their char yield are significantly improved to beyond 60 wt %, which are also much higher than that of the widely used carbon precursor, such as PAN (char yield ˜50 wt %). Thus, they are highly desirable as carbon precursors.

In order to assess mechanical performances of the products from the PN resins, 3-point-bending and DMA tests were studied as well. FIG. 5c shows the representative flexural responses of the PN-resin-based samples. The calculated modulus of PN-T-1 and PN-V-1 are 3.3 and 3.7 GPa with flexure strength of 156 and 135 MPa respectively. Additionally, the temperature dependence of storage modulus and tan δ were determined using DMA measurements and are as exhibited in FIG. 5d, whereas the maximum tan δ value is to evaluate their glass transition temperature (T_{g}). The initial storage modulus of PN-T-1 and PN-V-1 are 2.5 and 3.35 GPa at 30° C., which maintained up to nearly 200° C., showing their excellent capacity of thermal tolerance. Moreover, T_{g} of 280° C. and 295° C. are respectively found for PN-T-1 and PN-V-1.

Being post thermally treated, the PN based resins resulted in polymeric products with excellent thermal and mechanical performances. In particular, T_{g} of ˜300° C., T_{d} more than 400° C. and mechanical strength of 150 MPa were achieved. In consideration of the cyclisation process of nitrile groups as well as the polymerization of acrylate, a remarkably high cross-linking was established within these PN polymer network, which eventually led to their significant thermomechanical performances such as high storage modulus and T_{g}. These interesting findings of structural, thermal and mechanical properties are suggestive of the high-performance behaviours of the developed PN resins. The resins also exhibit significant high carbon yield, indicating that they are promising candidates as carbon precursors.

### Example 6. Digital Light Processing (DLP) Printing with PN-V-1 Resin Formulation, Post-Treatment and Carbon Conversion of GC Products

### Digital Light Processing (DLP) Printing

The DLP printing process was performed with a commercially available 3D printer with a UV light source of 405 nm. Computer aided design (CAD) of the print structures were designed in the software of Autodesk fusion 360 and the resulting STL files were sliced for a 2D file with different slicing thickness. After printing, the acquired objects were washed thoroughly with acetone to remove any residual resin, left to dry and then placed into a UV curing chamber for further photopolymerisation for 3 min.

### Post Treatment of Printed 3D Structures

The printed 3D structures were then placed into a vacuum oven at 50° C. for 24 h to remove residual solvent. Subsequently a progressive heat treatment was carried out by subjecting the samples to the following heating schedule: 120° C. (1 h), 160° C. (1 h), 180° C. (1 h), 200° C. (1 h), 220° C. (1 h), 240° C. (1 h) and 260° C. (12 h).

### Carbon Conversion of GC Products

The post-cured structures were subjected to heat treatment at 330° C. with 10° C./min and dwelt for 1 h, and then temperature was raised to 450° C. with ramping of 1° C./min and dwelt for 0.5 h, followed by temperature raising to 800° C. (or 1000° C.) with ramping of 1° C./min and dwelt for 1 h under a continuous purge of argon. Finally, the pyrolysed samples were naturally cooled to room temperature.

### Field Emission Scanning Electron Microscopy (FESEM)

The morphology of fractured surfaces of as-prepared samples was studied by field emission scanning electron microscope (FESEM) (JEOL JSM-7600F) and the sample was mounted on stainless holder for the SEM test.

### Raman Spectroscopy

Raman spectra were recorded by a Witec Alpha 300 SR spectrometer with an Argon ion laser (488 nm, 20 mW) as the excitation source.

### Results and Discussions

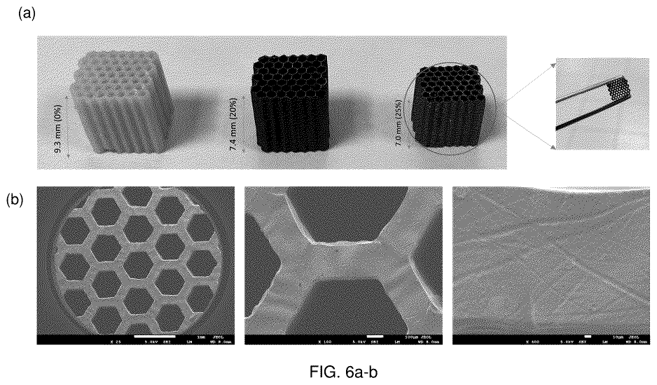

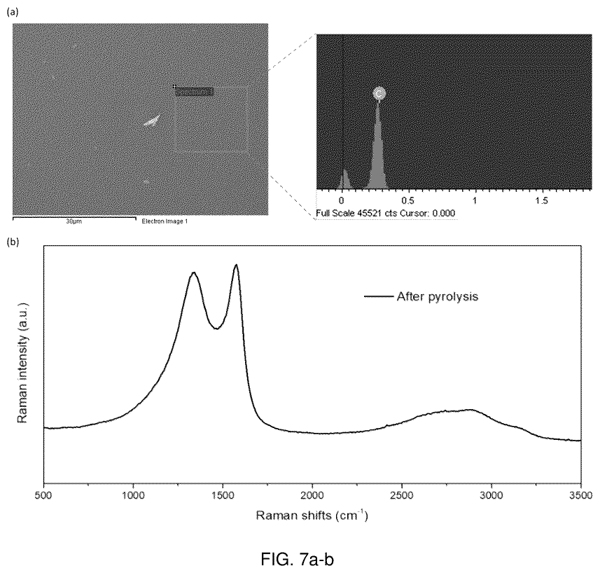

The formulated PN-V-1 resin was exposed to UV irradiation and generated a designed pattern with roughly 100 μm thicknesses. As the printing process was going on, structure geometry was built up in a layer-by-layer manner. Once the printing was completed, the structure was removed, further photocured and transferred to a second stage thermal curing. Eventually, pyrolysis was performed to obtain the 3D-printed GC products. FIG. 6a demonstrates the printed honeycomb structures at different stage. Measured by micrometre calliper, ˜25% of shrinkages are detected for the resultant GC structures. FIG. 6b exhibits the top morphology of the printed GC honeycomb structures using SEM measurements. As shown in the SEM screening of the cross-section of the GC structure in FIG. 7a, there are no detectable bubbles or voids on the fracture surfaces, thus suggesting a dense structure of the resulted GC object. Additionally, Raman spectrum further confirms the GC structure as shown in FIG. 7b. As DLP is a variant of projection micro-stereolithography (PμSL) 3D printing, these results show that the formulation based on PN-V monomers is suitable for PμSL 3D printing too.

### Example 7. PμSL Printing with PN-T-1 and PN-V-1 Resin Formulation, Post-Treatment and Carbon Conversion of GC Products

To confirm the above, PμSL printed structures as precursors were formed to generate 3D GC structures. The resins were based on the synthesized PN monomers formulated as described in Example 4. Characterisation by FESEM and Raman spectroscopy were done by following the protocol described in Example 6.

### Projection Micro-Stereolithography (PμSL) Printing

The PμSL printing process was performed with a commercially available PμSL 3D printer (nanoArch S140, BMF). A UV-LED (405 nm) was utilised as the light source. The intensity of 17.5 mW cm⁻² was used during all printing. During the printing process, the resin was exposed to a 405 nm light source for 8 s per layer, generating structured patterns with around 100 μm layer thicknesses. The exposure time and cured layer thickness were determined by prior curing depth measurements of the resins. Computer aided design (CAD) of the print structures were designed in the software of Autodesk fusion 360. The resulting STL files were sliced into a 2D file output based on the thickness determination. After printing, the acquired green body was carefully removed from the printing platform and washed thoroughly with acetone to remove any residual unreacted resin, placed into a UV curing chamber for further photopolymerisation for 6 min. and then transferred to remove solvent under vacuum for one day.

### Post Thermal Treatment of 3D-Printed Structures

The 3D-printed structures were then transferred to a vacuum oven to remove solvent and subjected to post-thermal treatment mentioned at the sample preparation part in Example 5.

### Conversion to GC Structures of the Printed Parts by Pyrolysis

The conversion from polymer to GC structures was carried out in a tube furnace with continuous argon gas flow. A progressive pyrolysis was utilised as the following: the thermally cured products of printed green bodies were subjected to 330° C. with a ramping of 10° C./min and dwelt for 1 h; then were heated to 450° C. with a ramping of 1° C./min and dwelt for 0.5 h; next, reached to the temperature of 800 or 1000° C. with 1° C./min and dwelt for 1 h. Finally, the pyrolysed samples were naturally cooled to room temperature.

### Determination of Density by Ethanol Displacement Method

The density of the GC that comprised the solid volume fraction of the honeycomb structure was determined by using ethanol displacement method.

### X-Ray Diffraction (XRD) Analysis

The X-ray diffraction (XRD) analysis was performed on a Bruker D8 Advance diffractometer with Cu-Kα radiation and 6-28 configuration.

### Compression Tests

Compression tests were measured on Instron 5567 frame equipped with 5 kN and 30 kN load cells at room temperature.

### Results and Discussions

As it was found that the resins have good 3D printing capability and can be fabricated using high-resolution PμSL additive manufacturing technology, complex 3D structured objects were herein printed and then subjected to post treatment. After being progressively pyrolysed to 1000° C., the printed objects can be readily converted into GC structures with structural integrity and fidelity (FIG. 9).

As the printing was going on, designed structure geometry was built up gradually in a layer-by-layer approach. Despite of usage of DMF solvent in the resin, layer shrinkage did not occur and complex 3D structures were generated with micrometer-scale resolution. As a result, complex 3D structured objects with dimensions around 10 mm were achieved with 100 μm layer thickness and 10 μm spatial resolution over print areas.

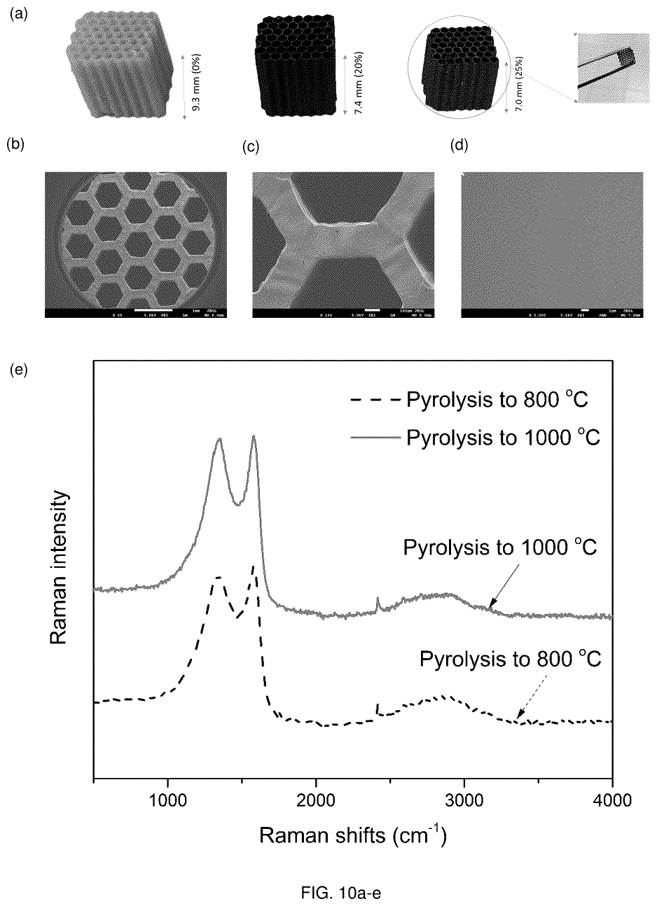

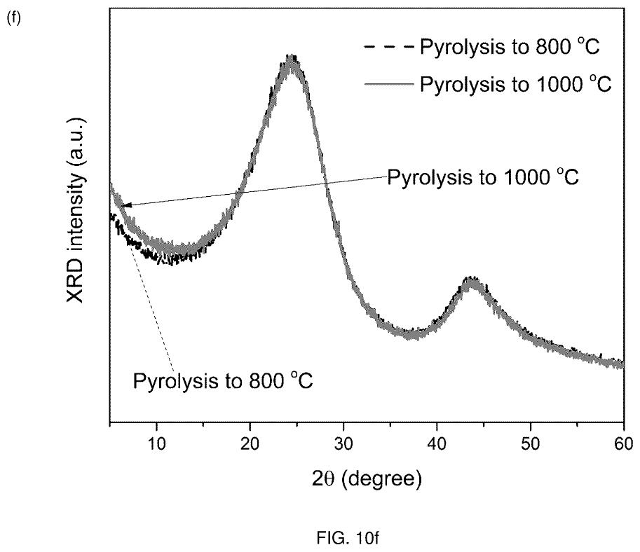

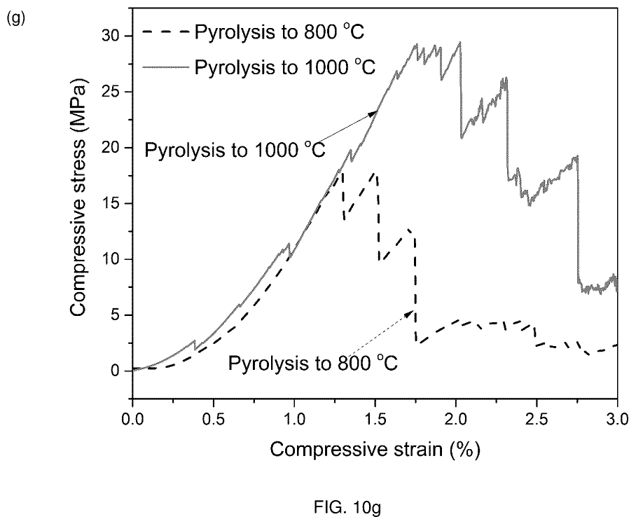

Conversion of 3D-printed PN based structures to GC consisted of two steps. In the first step, post thermal treatment was performed to generate highly cross-linked network to increase the mechanical rigidity and thermal stability of printed structures with the aim to maintain structural and geometric integrity during pyrolysis process. As shown in FIG. 10a, an obvious consequence of the post treatment of the as-printed structure (e.g., the honeycomb structure) is the colour change and the dimensional shrinkage. A dark colour and an isotropic shrinkage (20%) of the honeycomb structure occurred due to the cyclisation of nitrile groups and DMF solvent removal. Furthermore, there was no loss in structural integrity or part fidelity after the progressive thermal processing. The second step of GC conversion is the transformation of the cross-linked precursor to carbon via argon-induced pyrolysis to 1000° C. (or 800° C.) in a quartz tube furnace. A process with three isothermal heating stages was deployed to overcome cracking or disintegration of the thermally-treated printed objects during carbonisation. Following thermal treatments to 1000° C., the pyrolysed black objects presented shiny surfaces and exhibited an additional isotropic, linear shrinkage of ˜10% (FIG. 10a), resulting in a total of 25% shrinkage compared to the as-printed structure. This is due to the significantly high char yield of the PN resins. Furthermore, after pyrolysis, no cracks or pores were observed and the resultant objects remarkably retained geometric complexity and structural integrity.

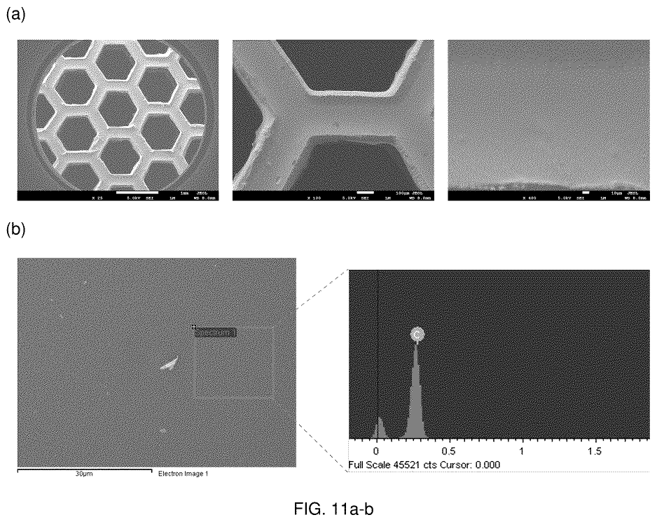

As displayed in FIG. 10b-d, scanning electron microscope (SEM) provided a visualisation of the morphology and structure of the as-printed as well as pyrolysed object ramping up to 1000° C. It can be seen that a regular honeycomb pattern was successfully produced during printing (FIG. 11a), suggesting the accuracy and precision of the employed PμSL technology. After pyrolysis, the objects maintained its structural integrity with clear and sharp-edged features and no structure deformation was found (FIG. 10b and FIG. 10c). Examination of the fractured objects with higher magnifications (FIG. 10d) revealed a dense and crack-free internal structure of the specimen. Despite the outgassing of hydrogenated and gaseous products during pyrolysis, the honeycomb specimen did not contain pores or internal cracking on both external and internal surfaces (up to 1000° C.), indicating the development of monolithic carbonaceous structure. Furthermore, elemental map-scanning of the pyrolysed internal surface suggests a high majority of carbon element of the pyrolysed object (FIG. 11b). It is known that one key parameter that differentiates GC from diamond or pyrolytic graphite is its density, which is generally between 1.4 and 1.8 g/cm³. The density of the carbonised honeycomb object (pyrolysed till 1000° C.) was determined by ethanol displacement method and it was found with a solid density of 1.62 g/cm³, suggesting its GC characteristic after pyrolysis.

Raman and XRD spectra were examined to assess the characterisation of the printed GC structures. In principle, Raman spectroscopy of carbonaceous materials provides information about microcrystalline carbon structure, such as the degree of ordering and crystallinity of carbon materials. Two key shifts are examined, which are the G band centred at 1580 cm⁻¹, representing crystalline graphite, and the D band centred at 1360 cm⁻¹, suggesting amorphous carbon structure and various kinds of defects in the graphitic structure (Amato, L. et al., Carbon, 2015, 94, 792; Beeman, D. et al., Matter Mater. Phys., 1984, 30, 870). As seen in FIG. 10c, the Raman spectra displays signature G and D bands, confirming the carbonaceous nature of the pyrolytic products derived from the PN resin. The breadth and intensity of the D band arises from the formation of a disordered carbon structure, which induces broadening of the G band and possibly causing the overlap of the D and G bands. It is also known that the ratio of the D and G bands intensity (I_{D}/I_{G}) is inversely proportional to the in-plane crystallite size. Herein, a larger I_{D}/I_{G} ratio corresponds to a smaller graphitic fraction and a higher disorder. In comparison to the final pyrolysis temperature of 800 and 1000° C., a very slight decrease can be found with the I_{D}/I_{G} ratio for the GC structures. Moreover, the broad 2D band at ˜2700 cm⁻¹, which originates from the presence of highly disordered and randomly arranged graphene with more than few layers (Hong, N. et al., Mater. Lett., 2012, 66, 60), shows that the intensity decrease as well, suggesting the formation of a more ordered carbon structure. As a result, higher pyrolysis temperature is able to influence on the microstructure and the graphitic content of the final GC products. X-Ray diffraction (XRD) patterns for the carbon pyrolysed at 800 and 1000° C. are shown in FIG. 10d. Both products exhibit similar diffraction pattern with broad peaks at 2θ˜23.5° and 2θ˜43.5°, corresponding to the (0 0 2) and (1 1 0) reflections from the carbon crystallites. The (0 0 2) peak corresponds to an approximate interlayer spacing of 3.8 A° (compared with 3.354 A° for ordered graphite), which is consistent with other XRD studies on vitreous carbon (Harikrishnan, G. et al., Carbon, 2007, 45, 531).

The quasi-static out-of-plane compression tests were performed to obtain the stress-strain curves using the carbonized honeycombs at a displacement rate of 0.1 mm/min. The representative stress-strain responses of carbonised honeycombs with different final pyrolysis temperatures are shown in FIG. 10e. It is clear that two curves both exhibit a brittle appearance. At the start of compression process, the compressive stresses of two specimens rise rapidly during elastic deformation stage before reaching the first initial buckling force values. The curve representing pyrolysis temperature at 800° C. shows a uniform growth trend until the stress value reaches close to 18 MPa, while the curve corresponding to the carbonised honeycomb with a pyrolysis temperature of 1000° C. occurred several small stress call-backs during the rapidly increasing elastic deformation stage until reaching the buckling point, which is close to 30 MPa. After this phase, the compression stresses drop quickly, and the stresses decrease step by step in a zigzag pattern until the stress value is zero. This is because the carbonised honeycomb structure has almost no plastic deformation during the compression process, thus the surface contacting the upper indenter collapses locally when the pressure reaches a certain value, and then other parts contacting the upper surface undertake the work under compression. When the pressure-bearing part reaches the limit again, it collapses. This process is repeated until the entire honeycomb is broken, and the stress value returns to zero when there is no pressure-bearing part at the contact position of the upper indenter.

The discrepancies among the stress-strain curves demonstrates that the pyrolysis temperature may significantly affect the compression behaviour of honeycomb structures. As seen from FIG. 10e, after the honeycomb is buckled, the buckling strength of the carbonized honeycomb with higher pyrolysis temperature is also higher, and in the process of subsequent collapse and energy absorption of the honeycomb, the time to failure of the honeycomb with higher pyrolysis temperature is also longer, where the location of the honeycomb failure is defined as the point where the compressive stress returns to zero. Then the compressive modulus of the two carbonised honeycombs were calculated from the linear fit of the two curves. The compression modulus of higher temperature pyrolysis carbonized honeycomb is 2.6 GPa, while the other is 2.4 GPa. As a result, when the pyrolysis temperature of the carbonised honeycomb is higher, both the compressive modulus and compressive strength are better, possibly due to the denser glassy carbon structure formed. However, because GC is very hard and brittle, its compressive strain is extremely small, and there is almost no plastic deformation during the compression process. There is only gradual collapse until the material fails and the energy absorption is very small.

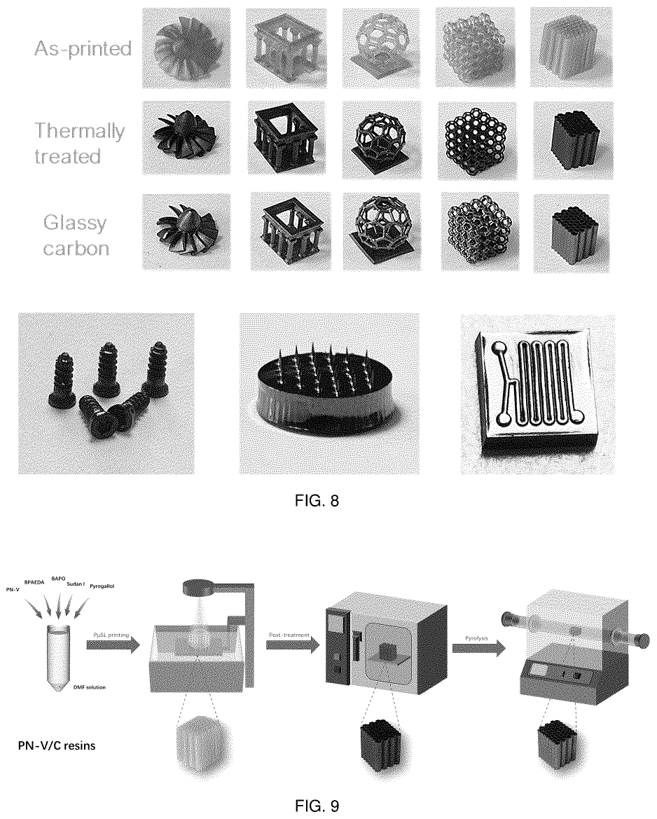

The good printability of the 3DPN resins and the pyrolysis conditions permitted the production of a wide variety of high resolution GC geometries. The efficacy of the process was demonstrated by fabricating structures of as-printed, thermally treated and final GC products using PμSL, as displayed in FIG. 8. All the obtained GC structures retained shape and structure integrity without cracks or voids. It has been reported that the opportunity to obtain micron-scale carbonaceous structures is potentially beneficial for the custom production of thermally robust structural components, customised electrical constituents, and lightweight structures that commonly comprise metal alloys.

### Example 8. Comparison of Printed GC Structures with Some Reported Results of Common Polymers for T_{g} and Carbon Yield

T_{g} and carbon yield were obtained by following the protocol described in Example 5.

### Results and Discussions

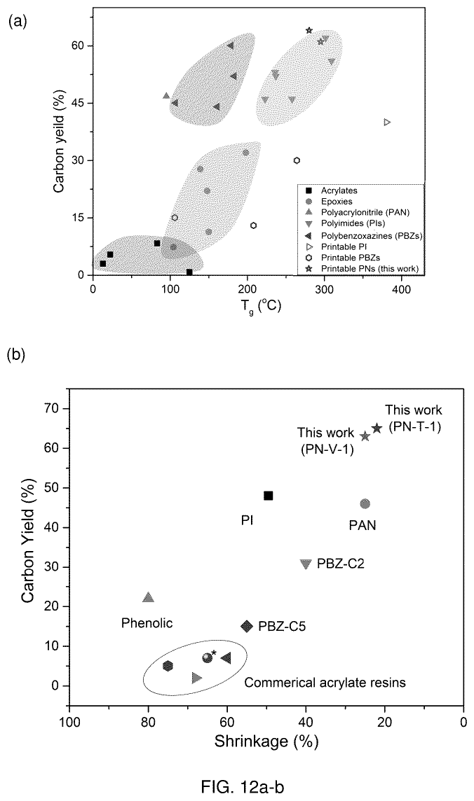

As shown in FIGS. 12a and 12b, compared to the commonly used plastic resins, PN-T-1 and PN-V-1 resins demonstrate remarkable T_{g} and high char yield associated with good photopolymerisable printability. This indicates the resins can result in high-performance products as well as being excellent promising carbon precursors. In addition, compared to printed carbons from other reported precursors, all resins demonstrate excellent char yield and ultralow shrinkage as shown in FIG. 12b, presenting their superior advantages on high-resolution GC printing.

This work not only expands the library of new class of high-performance PN resins for popular additive manufacturing but also features the precision 3D printing of GC with low shrinkage and high carbon yield for the first time.

1. A compound of formula I: 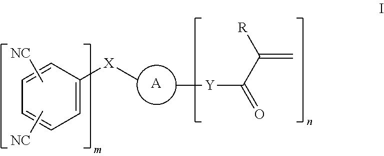
wherein:
m and n are independently from 1 to 3;
X is a bond, O or NH;
R is H or an aliphatic group; and
A is an aromatic, a heterocyclic or an aliphatic group and is unsubstituted or is substituted by one of more of the group consisting of halo, OR^{1a}, and NR^{1b}R^{1c};
R^{1a} to R^{1c} are each independently selected from H and C₁ to C₁₀ alkyl; and
Y is a bond, a C₁ to C₁₀ alkylene group or a
—C₁ to C₁₀ alkylene-O— group, where in the latter group the alkylene portion is directly attached to A and the oxygen atom is directly attached to the carbon of the C═O double bond.

2. The compound according to claim 1, wherein A is an aromatic or aliphatic group and is unsubstituted or is substituted by one of more of the group consisting of OR^{1a}, and NR^{1b}R^{1c}.

3. The compound according to claim 2, wherein A is phenyl and is unsubstituted or is substituted by one of more of the group consisting of OR^{1a}.

4. The compound according to claim 1, wherein R^{1a} to R^{1c}, where present, are H or CH₃.

5. The compound according to claim 1, wherein X is NH or O, optionally wherein X is O.

6. The compound according to claim 1, wherein n and m are 1.

7. The compound according to claim 1, wherein Y is a
—C₁ to C₃ alkylene-O— group, where the alkylene portion is directly attached to A and the oxygen atom is directly attached to the carbon of the C═O double bond.

8. The compound according to claim 1, wherein R is H or CH₃.

9. The compound according to claim 1, wherein the compound of formula I is selected from:

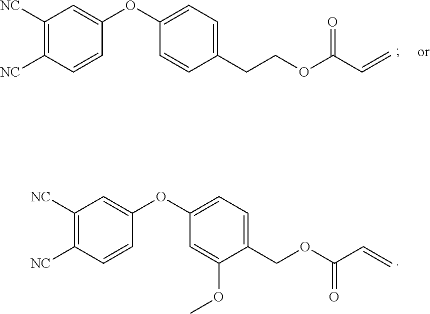 [SMILES: C=CC(=O)OCCc1ccc(Oc2ccc(C#N)c(C#N)c2)cc1 ; C=CC(=O)OCc1ccc(Oc2ccc(C#N)c(C#N)c2)cc1OC]

10. A resin precursor formulation for additive manufacturing, comprising:
a compound of formula I as defined in claim 1;
a photoinitiator; and
an organic solvent suitable for use in additive manufacturing.

11. The resin precursor formulation according to claim 10, wherein the formulation further comprises one or more of a crosslinking agent a photoabsorber, and a filler.

12. The resin precursor formulation according to claim 10, wherein the formulation is suitable for use in a stereolithography-type additive manufacturing process.

13. The resin precursor formulation according to claim 10, wherein one or more of the following apply:
(i) the photoinitiator is selected from one or more of the group consisting of 2,4,6-trimethyl benzoyldiphenyl phosphine oxide, 1-hydroxycyclohexyl phenyl ketone, 2-hydroxy-2-methylphenyl-propane-1-one, and, more particularly, phenylbis(2,4,6-trimethylbenzoyl)phosphine oxide); and
(ii) the organic solvent is selected from one or more of the group consisting of toluene, chloroform, tetrahydrofuran, and more particularly, toluene, dimethylformamide, and N-methylpyrrolidine.

14. The resin precursor formulation according to claim 10, wherein the resin precursor formulation comprises:
a compound of formula I;
wherein:
m and n are independently from 1 to 3;
X is a bond, O or NH;
R is H or an aliphatic group; and
A is an aromatic, a heterocyclic or an aliphatic group and is unsubstituted or is substituted by one of more of the group consisting of halo, OR^{1a}, and NR^{1b}R^{1c};
R^{1a} to R^{1c} are each independently selected from H and C₁ to C₁₀ alkyl; and
Y is a bond, a C₁ to C₁₀ alkylene group or a
—C₁ to C₁₀ alkylene-O— group, where in the latter group the alkylene portion is directly attached to A and the oxygen atom is directly attached to the carbon of the C═O double bond;
a photoinitiator;
an organic solvent suitable for use in additive manufacturing;
a crosslinking agent; and
a photoabsorber.

15. A polymeric resin composition obtained from the polymerisation of a compound of formula I as described in claim 1.

16. The polymeric resin composition according to claim 15, wherein the polymeric resin composition has one or more of the following properties:
(a) a T_{d} that is greater than or equal to 400° C.;
(b) a T_{g} that is greater than or equal to 300° C.;
(c) a bending strength that is greater than or equal to 100 MPa;
(d) a modulus greater than or equal to 3.5 Gpa;
(e) a carbon yield, following a sintering process, of greater than or equal to 60%; and
(f) a shrinkage value of from 20 to 40%, for a glassy carbon three-dimensional object relative to a green polymeric resin three-dimensional product following a sintering process to provide the glassy carbon three-dimensional product.

17. The polymeric resin composition according to claim 15, wherein the polymeric resin has been formed into a three-dimensional object.

18. A process of additive manufacturing, the process comprising:
(ai) providing a resin precursor formulation for additive manufacturing as described in claim 10; and
(aii) controlling a stereolithography-type apparatus to form a three-dimensional object by using the resin precursor formulation, wherein the resin precursor formulation is deposited by the apparatus on a surface in a layer by layer operation and each layer is subjected to a polymerisation reaction before each subsequent layer is laid down.

19. A process of providing a three-dimensional glassy carbon object, the process comprising the steps of:
(bi) providing a three-dimensional object comprising a polymeric resin material obtained from the polymerisation of a compound of formula I as described in claim 1; and
(bii) subjecting the three-dimensional object to carbonisation through the application of heat to provide a glassy carbon object.

20. (canceled)

21. (canceled)
